(19) United States
(12) Patent Application Publication
(10) Pub. No.: US 2018/0032691 A1
(43) Pub. Date: Feb. 1, 2018

(54) HEALTH MONITOR AND A METHOD FOR MONITORING HEALTH USING AN ARTIFICIAL INTELLIGENCE ENGINE PATTERN

(71) Applicants: Nissim ZUR, Givatayim (IL); Eli ARAD, Tzofit (IL)

(72) Inventors: Nissim ZUR, Givatayim (IL); Eli ARAD, Tzofit (IL)

(21) Appl. No.: 15/222,970

(22) Filed: Jul. 29, 2016

(51) Int. Cl. G06F 19/00; A61B 5/08; A61B 5/11; A61B 5/01; A61B 5/00; A61B 5/0402

(52) U.S. Cl. CPC G06F 19/3418; G06F 19/345; A61B 5/002; A61B 5/0402; A61B 5/1117; A61B 5/01; A61B 5/08; A61B 5/746

(57) ABSTRACT

A health monitor that includes a patch, a processor that is attached to the patch, a wireless transceiver circuit, one or more physiological sensors for sensing physiological information about a person when the patch is attached to the person, one or more additional sensors for sensing additional environmental information when the patch is attached to the person, and a memory unit; wherein the additional information is not physiological information; wherein the wireless transceiver circuit is configured to wireless transmit a health monitor identifier that identifies the health monitor and to receive, an artificial intelligence engine pattern associated with the person; wherein the memory unit stores the artificial intelligence engine pattern that (a) is at least partially based on to a medical history of the person; and (b) comprises combinations of values of physiological information and additional environmental information that represent events; and wherein the processor is configured to compare the physiological information and the additional information to the artificial intelligence engine pattern and to trigger alerts upon a detection of an event; and wherein the wireless transceiver circuit is configured to communicate the alerts.

### BACKGROUND

For early monitoring and diagnostic at people that are apparently supposed to be healthy. Somehow it is clear to anybody that vehicle's engine parameters like oil pressure and temperature are monitored constantly and needed to be done. But Humans are neglected in this sense of monitoring while been operating?!

### BACKGROUND OF THE INVENTION

Constitute significant public health burdens. Researches show that US$173 billion is spent every year for treatment of heart related disorders in USA^{[1]}. Atrial fibrillation (AF), a common arrhythmia, afflicts nearly 9% of persons over 80 years old^{[2]}, and is associated with increased stroke risk. Another arrhythmia, ventricular arrhythmia, can cause sudden cardiac arrest. Early diagnosis presents an opportunity for preventive treatment. However, many patients with cardiac arrhythmia or silent myocardial ischemia remain undiagnosed and untreated, because abnormal ECG changes often occur sporadically and are easily missed and neglected. Hence, a better ECG monitoring device is necessary.

The commonly used solutions like ambulatory Holter systems are often bulky with many wires stuck on patient's chest. The operational life of the Holter is limited within 72 hours, and ECG data are analyzed offline for diagnosis of problem. One major shortcoming of the existing ambulatory Holter systems is extremely low diagnostic yield at 10-13%^{[3]}. In addition, such devices use traditional ECG electrodes, which are not comfortable as there are multiple wires hanging around the body. And such devices usually aren't weatherproof; therefore the patient is expected to avoid water contact in the area where the device is fixed. All these, compromises patient's comfort level and affects his lifestyle.

A wireless ECG plaster device exists in several types. All can be used for real-time monitoring of ECG in cardiac patients. This device, when placed on patient's chest, continually records single-lead ECG and wirelessly streams it to a remote station for diagnosis. The skin contacts electrodes have been printed on flexible substrates with consideration for easy wearability. A highly integrated, low power chip with low noise amplifier, ADC and low pass filters were developed in-order to reduce the power consumption and the number of discrete IC components.

Legacy ECG plaster systems include two parts: a wireless ECG acquisition plaster, and a personal gateway (or remote station). The ECG plaster contains an ECG front-end chip, a microcontroller, and a wireless transceiver. The personal gateway can be either a mobile phone or a PC with an USB wireless interface. The plaster records the ECG and wirelessly transfers the data to remote data center through the personal gateway.

For example Legacy ECG plaster: The ECG acquisition chip is designed for low power use. For wireless communication, ZigBee or Bluetooth Smart (TI CC2420) is selected as it offers sufficient data rate at reasonable power consumption. The MCU (TI MSP430) is used for wireless baseband and for ECG data management. The plaster was designed with user comfort and ease of use in mind. Hence, it does not affect the daily activities of users. In addition, the plaster is sealed with splash and water-proof material, so the patient can take shower with the plaster.

At legacy wireless ECG plaster, the data is buffered using MCU internal memory before sending the gateway wirelessly. The maximum range of ZigBee or Bluetooth Smart transmission is about 5 to 15 meters in the room. The operational time is around few hours before battery fully discharge.

The firmware for ECG plaster performs the following tasks: ECG front-end and microprocessor initialization, managing ECG data buffering, and scheduling the wireless transceiver.

Since legacy wireless ECG plaster transmits its data wirelessly, all the time, it been limited to only 15 wireless channels as ZigBee transceiver. This also allows up to 15 patients to be monitored simultaneously.

Wi-Fi, Bluetooth classic, Bluetooth Smart devices work with the 2.4 GHz radio band, which is the same unlicensed frequency used by many other wireless devices. If many devices in the same area are all using the same stretch of bandwidth, it can lead to overall network problems, as the signals collide and information has to be resent. The wireless signal was designed to change its frequency many times per second to reduce this interference, but if enough devices are trying to use the same small stretch of bandwidth, interference is inevitable.

In addition, legacy wireless ECG plaster, due to power problems has function buttons or active or to deactivate the ECG sampling. This button is hard to reach by patient.

For a low-power weak-signal pickup device, one of the most essential links along the acquisition chain is its analog processing frontend and analog-to-digital interface. The required low noise, low distortion analog capabilities always conflict with the limited power budget. Unfortunately, such situation does not scale down with process technology as well as in digital domain, and in fact usually gets worse with more advanced process nodes.

The legacy wireless ECG plaster houses a fully featured bio-signal acquisition frontend, with all necessary tuning functions to cater for different input conditions. The front-end amplifier has on-chip high-impedance DC-blocking inputs that can be directly applied to ECG electrodes. The amplification stage consists of a low noise front-end amplifier with band-pass function and a programmable gain amplifier (PGA) employing the flip-over-capacitor technique^{[4]}, both op-amps are biased in sub threshold mode to ensure optimal noise efficiency against power. During startup or after an input interruption event such as electrode falloff, a reset signal is asserted to eliminate the large time constant associated with the high-pass filter, such that the preamplifier can quickly resume operation. A series of secondary low-pass filters then provides further suppression to the out-of-band residues such that lower sampling frequency (in this case 3 times of signal bandwidth for over 20-dB attenuation) that favors lower wireless bit rates can be used. Following the analog processing modules, a 12-bit charge redistribution SAR ADC quantizes the conditioned ECG signal based on the sampling speed set by the microcontroller, and encodes the data into 16-bit SPI frames.

### SUMMARY

There may be provided a health monitor that may include a patch, a processor that is attached to the patch, a wireless transceiver circuit, one or more physiological sensors for sensing physiological information about a person when the patch is attached to the person, one or more additional sensors for sensing additional environmental information when the patch is attached to the person, and a memory unit. The additional information is not physiological information. It may be biomechanical information, acceleration, speed, posture information and/or ambient information such as location, altitude, ambient temperature, humidity and the like. The wireless transceiver circuit is configured to wireless transmit a health monitor identifier that identifies the health monitor and to receive, an artificial intelligence engine pattern associated with the person. The pairing between the person and health monitor identifier may be done by the person, a device (such as a smartphone) accessible to the person or by any other means). The health monitor not have the capability to calculate the artificial intelligence engine pattern—which reduces the cost of the health monitor—and may use could computing or any other device that differs from the health monitor to calculate the artificial intelligence engine pattern. The term artificial intelligence may mean intelligence (see Wikipedia) exhibited by machines. In computer science, an ideal “intelligent” machine is a flexible rational agent that perceives its environment and takes actions that maximize its chance of success at some goal. Colloquially, the term “artificial intelligence” is applied when a machine mimics “cognitive” functions that humans associate with other human minds, such as “learning” and “problem solving. The term artificial intelligence may refer to an algorithm that may apply learning skills on multiple types of information (such as physiological information, additional information and person's medical history). The memory unit (such as flash or non-flash memory unit) may store the artificial intelligence engine pattern (also referred to as pattern) that (a) is at least partially based on to a medical history of the person; and (b) comprises combinations of values of physiological information and additional environmental information that represent events. The processor is configured to compare the physiological information and the additional information to the artificial intelligence engine pattern and to trigger alerts upon a detection of an event. The event may represent an undesired combination of information. The wireless transceiver circuit may be configured to communicate the alerts. The alerts may be communicated, directly or indirectly, to a more powerful computerized system (such as cloud computer, server, desktop computer, non-cloud computer)

The at least a majority of a transmissions of the wireless transceiver circuit are the alerts.

The additional information may include a location of the person.

The additional information may include a detection of an event that includes sensing a gesture of the person.

The health monitor according to claim 4 wherein the gestures triggers a communication with a call center.

The wireless transceiver circuit may be Bluetooth smart compliant.

The wireless transceiver circuit may be configured to transmit the physiological information and the additional aggregated stored information.

The physiological information may be about ECG activities, movements, instability, falling, heat, respiration.

The wireless transceiver circuit may be configured to sense and store in the memory transmissions from wireless identification cards of nurses and doctors.

The health monitor may be configured to monitor the health monitor battery level and fire alert on low levels.

The health monitor may be configured to detect patterns of known cardiovascular diseases. In this case the artificial intelligence engine pattern will reflect combinations of physiological information and/or additional information that are indicative of these known cardiovascular diseases.

The health monitor may be configured to detect different person's daily activities and use the right patterns to the activity.

The health monitor may be configured to monitor the physical information and the additional information while the person performs heart stressing activities and report the physical information and the additional information to an external device/

The one or more physiological sensor are non-invasive sensors.

There may be provided a method for health monitoring, the method may include: transmitting by a wireless transceiver circuit of a health monitor to a communication device a health monitor identifier that identifies the health monitor; receiving, in response to the health monitor identifier, an artificial intelligence engine pattern associated with a person; sensing physiological information of a person by one or more physiological sensors while a patch may be attached to the person; sensing by one or more additional sensors additional information about the person while the patch may be attached to the person, the additional information may be not physiological information; storing in a memory unit the artificial intelligence engine pattern that (a) may be at least partially based on to a medical history of the person and (b) provides alert triggering combinations of values of physiological information and additional information; comparing, by a processor that may be attached to the patch, the physiological information and the additional information to the; triggering, based on the comparing, alerts; and communicating the alerts by the wireless transceiver circuit.

There may be provided a method for health monitoring comprising: calculating, at least partially based on a medical history of a person, a pattern that (a) may be at least partially based on to a medical history of the person and (b) provides alert triggering combinations of values of physiological information and additional information; participating in sending the pattern to a health monitor that may be associated with the person; receiving from the health monitor, new physiological information of the person and new additional information of the person; updating the pattern to provide an updated pattern; and participating in sending the updated pattern to the health monitor.

### BRIEF DESCRIPTION OF THE DRAWINGS

FIG. 1 shows the Artificial Intelligence building blocks units, as system architecture, that serves and delivers data to the Artificial Intelligence algorithms, to auto learning and auto decision, if the vital signs need an immediate wireless alert;

FIG. 2 shows the Artificial Intelligence vital signs auto alert wireless plaster position on the human body and placements of its Hardware part—PCB;

FIG. 3 shows the Artificial Intelligence algorithm detecting different patient's daily activities. With this info and data from the sensors it detects if there is a sign that can fire wireless event;

FIG. 4 shows the Artificial Intelligence algorithm detecting different patient's daily activities. With this info and data from the sensors it detects if there is a sign that can fire wireless event;

FIG. 5 shows the Artificial Intelligence algorithm detecting different patient's daily activities. With this info and data from the sensors it detects if there is a sign that can fire wireless event;

FIG. 6 shows artificial intelligence vital signs wireless plaster “machine decision table” for early detection, prediction and Prevention;

FIG. 7 illustrates the Brugada syndrome is a genetic disease;

FIG. 8 shows ECG Electrocardiography;

FIG. 9 shows ECG Abnormalities; and

FIG. 10 shows the Artificial Intelligence algorithms and pattern detection of ECG QT interval for Cardiovascular Disease early detection, predicted and Prevention

### DETAILED DESCRIPTION OF THE DRAWINGS

Any reference to a method should be regarded as a reference to a system and/or device for executing the method and to a computer program product (which is non-transitory and stores instructions that once executed by the health monitor or any other computerized system results in the execution of the method). The same applies to the system and/or device and the computer program product.

Any combination of any method step and/or system and/or device and or/health monitor component may be provided.

The term patch may refer to the material and/or element that is detachably attached to the person and/or to the entire health monitor.

There may be provided apparatuses, methods, computer program product and systems for an artificial intelligence vital signs wireless plaster for Cardiovascular Disease early detection, prediction and Prevention simultaneously detects patient's temperature, respiration rate, Blood oxygen monitor, elderly people evolving problems as movements, instability, falling etc.

The proposed device is a very light weight plaster, attached to the chest. It has an ability to detect hand proximity to the plaster through clothing and send alert to smart phone or any wireless recipient. It can wirelessly transmits, while on an “alert event”, the patient's ECG signal with all data stored from other sensors, to mobile phone or to wireless mesh device around it.

The device runs several processes in parallel that store all data from sensors and in parallel by an Artificial Intelligence engine, all aggregated sensors data, patient's history from internet, Aggregates patient's current activities as sleeping, walking, exercising, with gathered data and setting from the internet and deciding when to fire the “event”.

The Artificial Intelligence saves the aggregated data into device's flash storage and updates the patient's data base on the internet.

The device transmits wirelessly data only on Artificial Intelligence events that extends the battery life immensely and prevents RF radiation of the patient.

The present invention relates to an improvement for Problems exposed with the legacy wireless ECG plaster Prior art and an improvement over legacy holter.

Artificial Intelligence vital signs auto alert wireless plaster provides services to the patient as a human doctor does. Retrieves patient's medical history and patient's setting from the internet and serves as wireless alert system that detects any variant from patient's medical history. The Artificial Intelligence engine monitor ECG activities, movements, instability, falling, heat, respiration and patient's hand over the plaster to fire an Event to “call center”, using patient's smart phone or fix location Bluetooth Smart Wi-Fi bridge. On an alert event, the Artificial Intelligence sends all stored data of old sensors vital signs and the cause of immediate alert. For example ECG non-normal activities, high temperature, low oxygen level in blood, low respiration rate, falling, tremor, sleep appena etc.

The suggested health monitor does not perform nonstop wireless transmission consumes the device's battery and limits its use.

The suggested health monitor does not perform nonstop wireless transmission—thereby it does not limit the number of devices in same area as hospital rooms and also reduced radiation.

The suggested health monitor may be configured to monitor health and generate an alert upon an occurrence of an event.

The suggested health monitor may include an artificial intelligence engine that aggregates information from patient history, current activities, skin temperature and starts wireless transmission (only) on an “event”

Artificial Intelligence vital signs auto alert wireless plasters electrical functions:

1. wireless connection

2. low power CPU and sensors

3. High sensitive programmable, clinic grade ECG

4. Supports Internet of Things (“IoT”) wireless Bluetooth Smart with mesh to any distance,

and bridges to internet retrieving patient's medical history and setting and storing patient's vital signs, and retrieve commands.

5. A wireless location based detection using smart phone or Bluetooth Smart Wi-Fi bridges

6. A temperature sensor

7. Highly sensitive 3D accelerator. Respiratory rate, day/night exercise

8. Highly sensitive pressure sensor that provides altitude data. (30 cm resolution)

9. Proximity Panic button. A proximity alert sensor. Active through clothing up to 1.5 cm depth. No physical buttons.

10. Blood oxygen monitor

11. Digital flash storage for several months. A parallel Holter mode for all vital signs.

12. A unique 2 processors in one package Very fast parallel processing for Artificial Intelligence algorithm, to automatic decoding alert on ECG or body vital signs, as shaking falling, etc.

13. Water prof. Can be stick to skin for full week for early diagnostic of subtle signs of heart evolving problems.

14. Very low cost, around 5 USD, possibility for disposable.

15. Auto detects alert state, can flash led, sounds a buzzer, parallel to auto wireless link to support center.

An artificial intelligence vital signs wireless plaster for early detection of:

1. Prediction and Prevention cardiovascular Disease early detection,

2. Prediction and Prevention people in risk of falling. As elderly people,

3. Prediction and Prevention respiration problems as sleep apnea,

4. And other Artificial Intelligence detecting out comes of the sensors on the plaster to enhance medicine diagnostics.

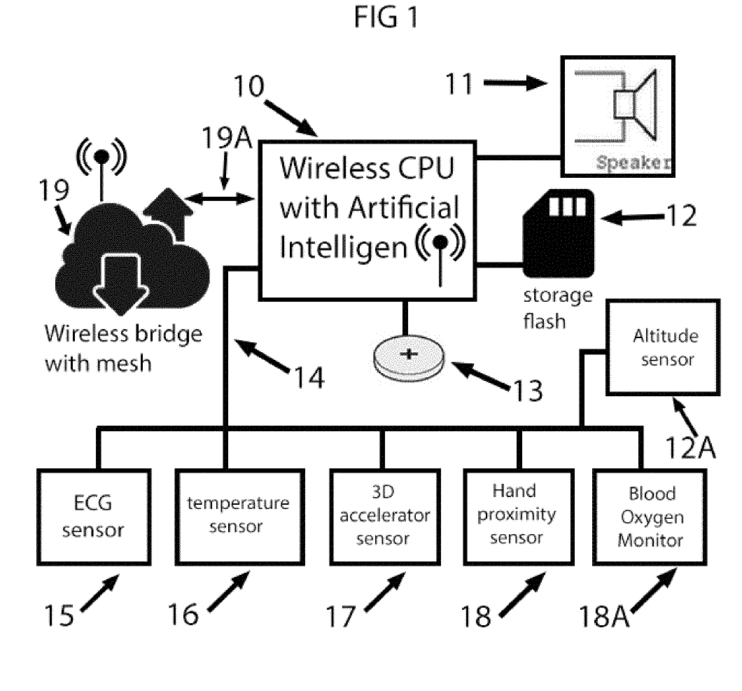

FIG. 1 shows the Artificial Intelligence building blocks units as system architecture that serves and deliver data to the Artificial Intelligence algorithms to auto learning and auto decision if the vital signs need immediate wireless alert.

FIG. 1 reference number 10 is a Dual processing CPU, as CSR1010 that can handle from one processor the wireless connection to internet, to retrieve patient's medical history and upload stored data, and second processor run Artificial Intelligence detecting alert situation. FIG. 1 reference number 10 has serial bus to sensors, as I2C UART and SPI (FIG. 1 reference number 14), a connection to local battery (FIG. 1 reference number 13).

FIG. 1 also shows (see reference number 12A) a high resolution altimeter (Altitude) sensors I2 C bus interface. This altitude pressure sensor is optimized for fast changes with an altitude resolution of 30 cm. The sensor module includes a high linearity pressure sensor and an ultra-low power 24 bit ΔΣ ADC with internal factory calibrated coefficients. It provides a precise digital 24 bit pressure and temperature value and different operation modes that allow the Artificial Intelligence detecting the height of the plaster. If the patient falls, it detects it also by this height. It uses also to detect when the user is lying in bed and not on the floor. Most altimeters can provide a fix height from sea level due to electronic barriers that deliver Accuracy 25° C., +−1.5 mBar, as MS5607-02BA03. But the Artificial Intelligence does not use the absolute height as a reference, and instead uses aggregate data reading differential height and changes within few second to detect falling on the floor or lying in bed. The Artificial Intelligence also collects streaming data from (FIG. 1 reference number 17) 3D accelerometer, and with the altitude sensor can determine patient's status. At bed, falling on the floor or any other body activities.

The Artificial Intelligence commenced also with FIG. 1 reference number 15 to collect streaming ECG data. Also collects streaming data from (FIG. 1 reference number 16_skin temperature. and alerts on high temperature. or on rare cases of low temperature (hypothermia).
It has input also from (FIG. 1 reference number 18) hand proximity detector, and when user's gesture on it, as taping through the clothing, the Artificial Intelligence algorithm, overrides self-detection and fire a wireless alarm immediately.

On an Alarm situation, the CPU activates the local speaker/buzzer to confirm and advise patient that an alert has been sent. A wireless connecting (FIG. 1 reference number 19A) is made over patient's smart phone or fix location room Bluetooth Smart-Wi-Fi bridge. The smart phone sends a call center over the internet the alert with patient's stored data from the flash (FIG. 1 reference number 12) and the phone GPS fix location. The Bridge FIG. 1 reference number 19) does the same with its MAC address, in a-way that the call center can detect it and knows its physical location.

The Artificial Intelligence retrieves also information from the blood oxygen monitor (FIG. 1 reference number 18A) and fire an alert on low oxygen in the blood.

(FIG. 1 reference number 19A) the wireless bridge can be any smart phone or fix location devices that can communicate from one side with the plaster wirelessly, and from the other side with the internet.

This link is used for:

1. Immediate alert on Artificial Intelligence detects cause an alert event.

2. Sends stored sensors data from plaster local flash to internet data base

3. Retrieves patient's medical data base to be used by the plaster AI with real time info from sensors.

4. Retrieves commands and setting from patient's doctor and care giver

5. Retrieves a new algorithms to the Artificial Intelligence to detect new alert pattern events

6. Retrieves experimental data to be tested with the Artificial Intelligence algorithms instead of real time sensor's data.

7. Senses location by proximity to wireless bridge or smart phone GPS and sends to internet.

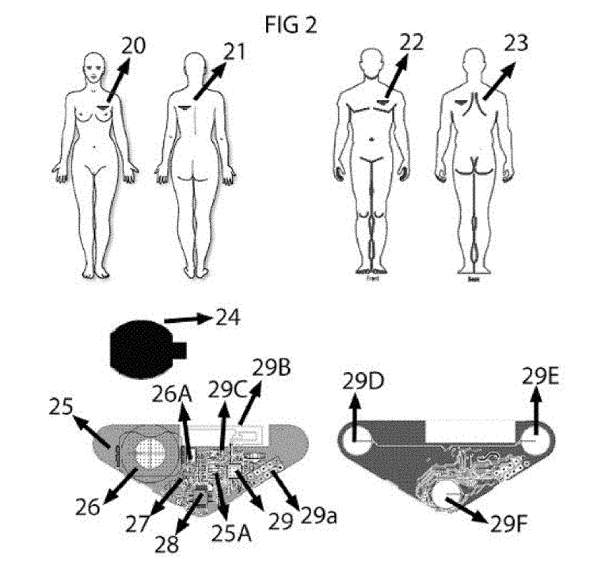

FIG. 2 Shows the Artificial Intelligence vital signs auto alert wireless plaster position on the human body and placement of its Hardware part—the PCB. The plaster can be placed on the front chest (FIG. 2 reference numbers 20 and 22) or on the back (FIG. 2 reference numbers 21 and 23) to prevent from mental ill patients to remove it.

FIG. 2 reference number 24 is a metal PCB pad that is placed above the battery to detect patient's hand hovers over it, to activate an alarm. It detects unique pattern to prevent fault activation, as non unique taping. The Artificial Intelligence detects that patient's request and fire an alarm. FIG. 2 reference number 5 is above the ECG probe. It is covered will full ground, 3 layers, to shield the ECG probe on the bottom to revised RF interfering from the wireless side of the plaster or other sources.

FIG. 2 reference number 26 is the battery place. It used to power the battery. The shape can be changed to flat one too.

FIG. 2 reference number 27 is the place of the proximity sensor.

FIG. 2 reference number 28 is the ECG chip.

FIG. 2 reference number 25A is the speaker buzzer attaching point. It is placed above the PCB below the antenna

FIG. 2 reference number 29 is the 3D accelerometer. It used to detect body movement and chest movement as respiration sensor too

FIG. 2 reference number 29A is the location of connector to insert code into the CPU.

FIG. 2 reference number 29B is the PCB antenna for wireless communication.

FIG. 2 reference number 29C is the location of Storage flash the location of Blood Oxygen sensor and the Altitude sensor

FIG. 2 reference number 26A is the wireless CPU with temperature sensor

FIG. 2 reference number 29D is the plaster bottom side left ECG probe. It is part of the PCB and has a conductive sponge.

FIG. 2 reference number 29E is the plaster bottom side right ECG probe. It is part of the PCB and has a conductive sponge.

FIG. 2 reference number 29F is the plaster bottom side “left leg” ECG probe, the reference GND for the ECG chip. It is part of the PCB and has a conductive sponge. All other areas of the plaster are covered by solder mask and sink on silicon to prevent of water leaking in.

The probe sponge can be wet and it doesn't affect the ECG sensor reading due to Artificial Intelligence algorithm that detects wet condition and filter that from the ECG stream of data.

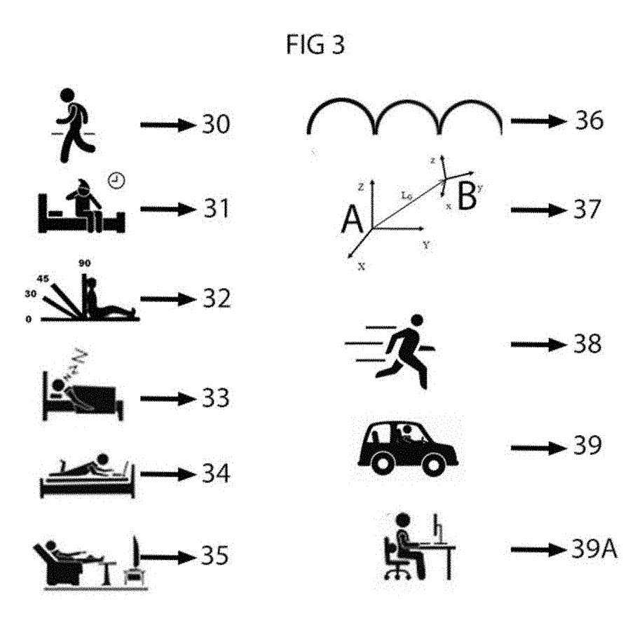

FIG. 3 Shows the Artificial Intelligence algorithm for detecting different patient's daily activities. The Artificial Intelligence works with multistage streaming retrieval and storage while examining all data from sensors and historical data and tries to find known patterns to determine the patient's body activity.

Knowing the patient's body activity is the first stage of multi stage actions to be recognized an alert situation.

For example:
High ECG rate and high respiration rate while patient's is OK on basketball play. But needs an immediate alert if it sleeps.
Low or no chest movement in sleeping is an immediate alert. But no alert if it takes a shower. In this case the accelerometer, the altitude and ECG process by the Artificial Intelligence algorithm.
Etc.

With all sensors streaming data, recognizing patient's activities the Artificial Intelligence “decided” to fire wireless event.

Here are parts of the patient's body detections, and what sensor was used for that:

FIG. 3 reference number 30 detects that the patient is walking. The 3D accelerometer reports horizontal movement as with small velocity up and forward as of normal walking. The accelerometer stream of data is stored over time and old data is been used to know the present situation (FIG. 3 reference number 37). So when user goes to bed, the Artificial Intelligence detects walking (FIG. 3 reference number 36) and siting on bed (FIG. 3 reference number 31). Siting on bed is detected since the altitude sensor reports lower 0.5 meter, and the ACC reports small velocity toward earth. When patient lays back on bed (FIG. 3 reference number 32) The Artificial Intelligence detects this data from the accelerometer and altitude sensor.

Sleeping state (FIG. 3 reference number 33) is one or states of the Artificial Intelligence vital signs auto alert wireless plaster. Since the Artificial Intelligence needs to fine tune, it alerts sensing as stop chest movement, high ECG, lower oxygen in blood. As sleep apnea symptoms.

The Artificial Intelligence detects the restless sleep as in sleep apnea symptoms. This is very serious since it can cause sleepiness while driving, Sleepiness or lack of energy during the day, Sleepiness while driving, Morning headaches, Restless sleep, Forgetfulness, mood changes, and a decreased interest in sex, Recurrent awakenings or insomnia.

Also, in sleeping, the Artificial Intelligence detects rising of the temperature, ECG is going high without any real body movement etc.

The Artificial Intelligence can detect also state of in the bed but not sleeping (FIG. 3 reference number 34).

On the sofa (FIG. 3 reference number 35) the accelerometer reports back—“not horizontal”. The altitude sensor reports the height of standing.

FIG. 3 reference number 38 illustrates patient's state of running. The accelerometer reports high velocity up and front forwards, with chest tilted forward. So it is not alert state if ECG, respiration, and skin temperature go high.

But if that is too high, an alert will be fired. As a marathon runner dies due high ECG and skin temperature.

FIG. 3 reference number 39 illustrates a state of driving. The accelerometer reports high velocity forward without pedometer FIG. 3 reference number 39 pattern. It is important to detect a heart problem and report it to car eHealth center to stop the car.

FIG. 3 reference number 39A illustrates a state of siting at a desk work, and reminds the user to sit straight up and walk around or have an activity done from time to time.

FIG. 3 reference number 38 illustrates a state of falling back down the stairs. The Artificial Intelligence gets this info from accelerometer and altitude sensor. Very important for elderly people.

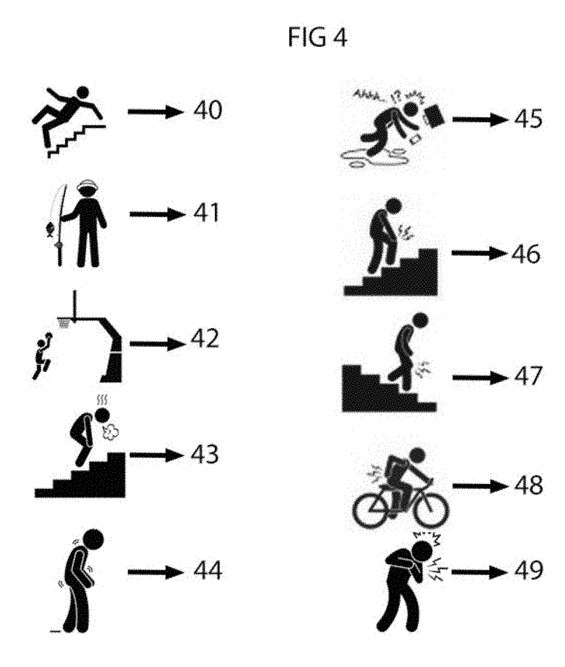

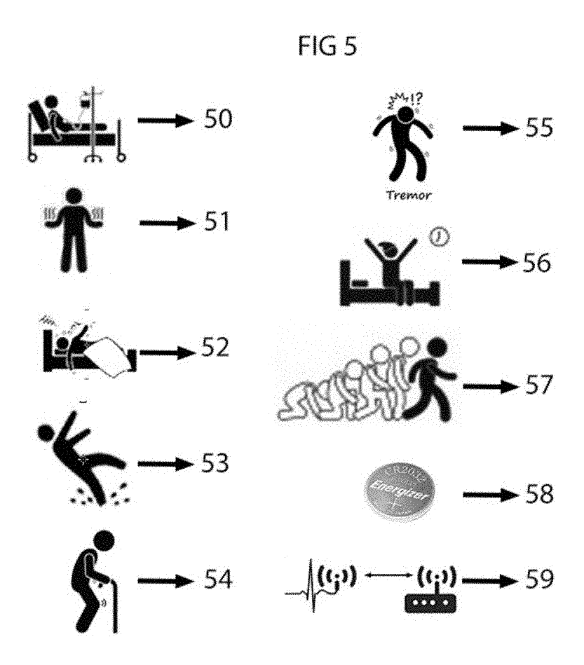

FIG. 4 reference number 40 illustrates a state of falling back down the stairs. The Artificial Intelligence gets this info from accelerometer and altitude sensor. Very important to elderly people.

FIG. 4 reference number 45 illustrates a state of falling forwards. The Artificial Intelligence gets this info from accelerometer and altitude sensor. Very important to elderly people.

FIG. 4 reference number 41 illustrates a state of minor body action, as fishing.

FIG. 4 reference number 46 illustrates a state of tremor while climbing the stairs. Very important to elderly people for early detection before falling and braking bones.

FIG. 4 reference number 47 illustrates a state of tremor while down the stairs. Very impotent to elderly people for early detection before falling and braking bones

FIG. 4 reference number 43 illustrates a state of high chest activity and pausing while climbing the stairs. Very important for elderly people to early detection general degradation of health before heart stroke or falling and breaking bones

FIG. 4 reference number 44 illustrates a state of different tremor of walking pattern from old historical data. Very important for elderly people to early detection general degradation of health before heart stroke or falling and breaking bones

FIG. 4 reference number 42 illustrates a state of sport activity. Data collected from accelerometer and altitude sensors. And compare by old historical data. Important to prevent flash alert, and show to general energy spending.

FIG. 4 reference number 48 illustrates a state of bicycle sport activity. Data collected from accelerometer and altitude sensors. And compared by old historical data. Important to prevent flash alert, and show general energy spending.

FIG. 4 reference number 49 illustrates a state of heart attack event. The Artificial Intelligence detects the non-usual ECG and automatically fire an alert by wireless and immediately with stored data and GPS position information. The user's smart phone is used for bridging the wireless data from the plaster to the internet. The GPS location is retrieved from the phone.

FIG. 5 reference number 50 illustrates A patient that is lying at bed at the hospital. The plaster eliminates the need for wires to hospital ECG. Eliminates the need for respiration monitor, eliminates the need for blood Oximeter. A wireless alert proximity button is added to call the nurse.

If a patient is not allowed to get off the bed by himself, by trying to do that it may alert the nurse, before the patient may fall.

If the patient feels bad while taking a shower or at any place along the hospital's corridor, he just has to hover his hand on the proximity alarm button and a wireless alarm with his locating, will be sent/“fire”. The patient's location can be detected in every room or hall in the hospital.

Assuming nurses and the doctors will wear a wireless ID card. The plaster can detect proximity to hospital staff, and store that info in its flash memory, so it will be known who was taking care of the patient and in what date/time. All of this data can be wireless upload to internet or hospital information center. The Artificial Intelligence vital signs auto alert wireless plaster can detect proximity to other wireless plaster, to a fix locating bridges and to hospital staff wearing wireless badges.

The hospital staff of patient's doctor can update setting in the plaster regarding margins to an immediate alert. It can be done by smart phone, PC, tablet or automatically from hospital information center on real time. For example, after a surgery the patient does not allowed to go off the bed by himself, but after 2 days that rule is automatically changed.

FIG. 5 reference number 55 illustrates the Artificial Intelligence detects sudden tremor and fire an alert.

FIG. 5 reference number 51 illustrates the Artificial Intelligence detects sudden high temperature and fire an alert.

FIG. 5 reference number 56 illustrates the Artificial Intelligence detects if a patient did not go out of bed in the morning, as an event of a problem in elderly people that live by themselves.

FIG. 5 reference number 52 illustrates the Artificial Intelligence detects abnormal movement in bed as an event of problem in elderly people that live by themselves. Or some sleep apnea symptoms.

FIG. 5 reference number 57 illustrates the Artificial Intelligence detects falling without injury since the patient continues to walk after that. No need for wireless alarm.

FIG. 5 reference number 53 illustrates the Artificial Intelligence detects falling back—immediately wireless alarm.

FIG. 5 reference number 58 illustrates the Artificial Intelligence detects battery level and send wireless alarm before battery will be dead.

FIG. 5 reference number 54 illustrates the Artificial Intelligence detects changes in walking pattern in elderly people and alert before falling.

FIG. 5 reference number 59 illustrates the Artificial Intelligence detects distance to Smart phone, wireless bridge or hospital staff personal and store the info in internal plaster flash (Figure reference number 12) and use the wireless nearest connection to send wireless alarm on event.

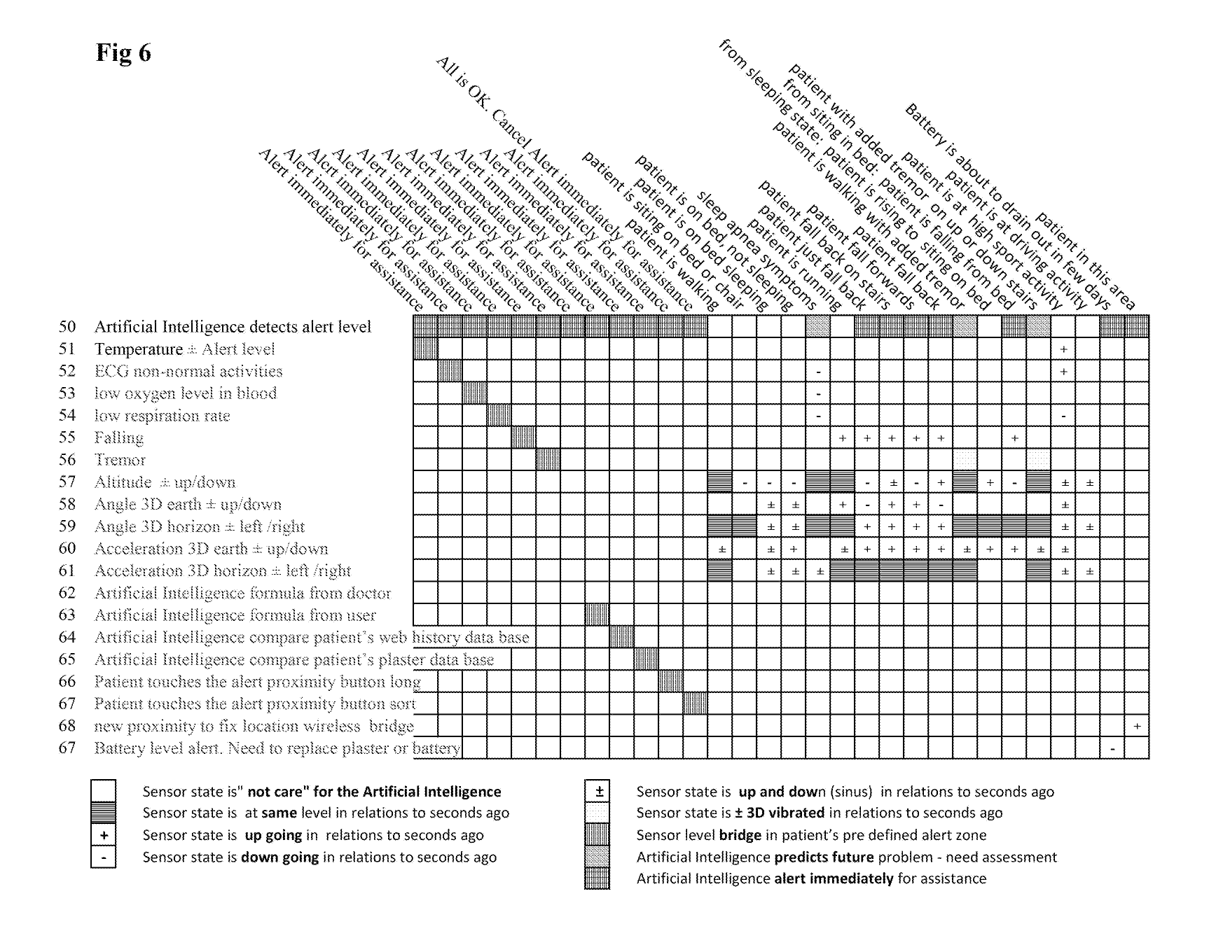

FIG. 6 illustrates an artificial intelligence engine pattern.

FIG. 6 shows, for example, an artificial intelligence vital signs wireless plaster “machine decision table” for early detection, prediction and Prevention for the following targets:

1. Cardiovascular Disease early detection, Prediction and Prevention

2. People at risk of falling. as elderly people

3. Respiration problems as sleep apnea

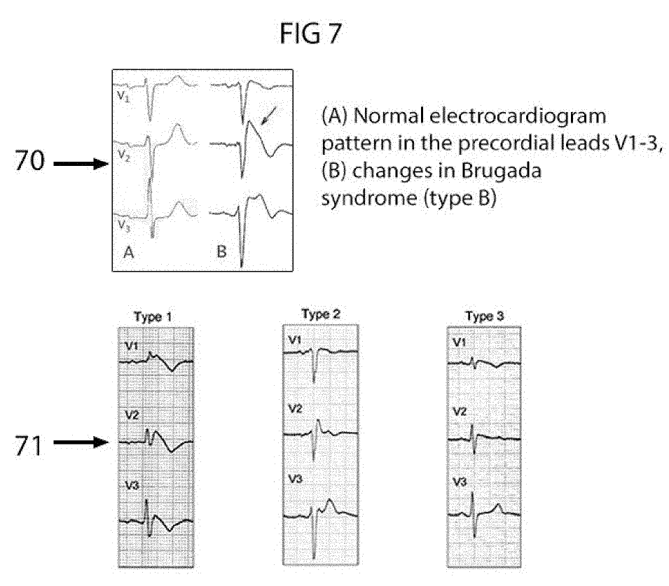

FIG. 7 reference number 70 illustrates The Brugada syndrome is a genetic disease. The Artificial Intelligence detects known patterns to detect known ECG and Respiration syndromes. This is a one example of the detection syndrome. Other syndromes patterns detected too in the same way. The Brugada syndrome is a genetic disease that is characterized by abnormal electrocardiogram (ECG) findings and an increased risk of sudden cardiac death. It is named by the Spanish cardiologists Pedro Brugada and Josep Brugada. It is the major cause of sudden unexplained death syndrome (SUDS), also known as sudden adult death syndrome (SADS), and is the most common cause of sudden death in young men without known underlying cardiac disease in Thailand and Laos.

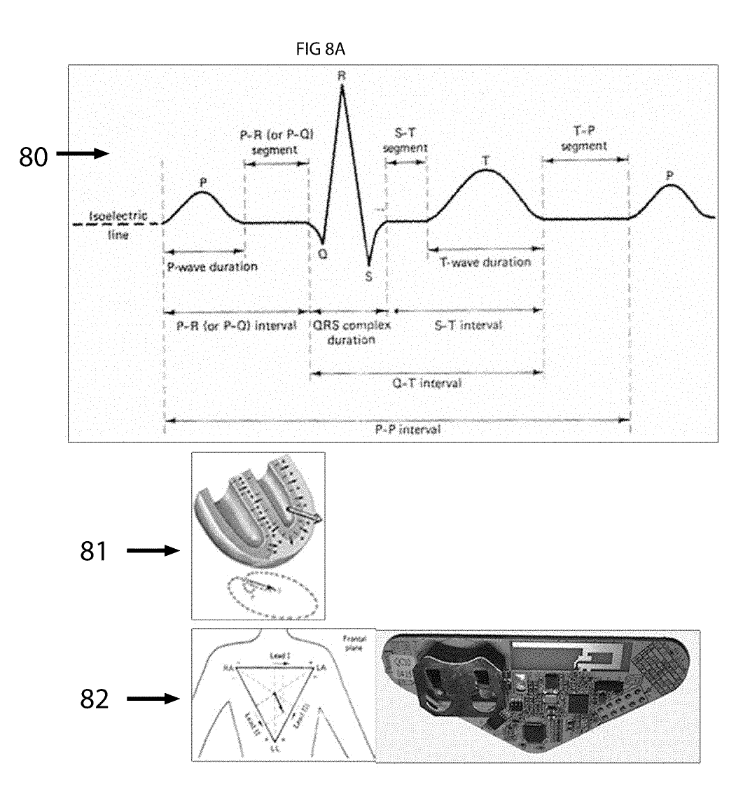

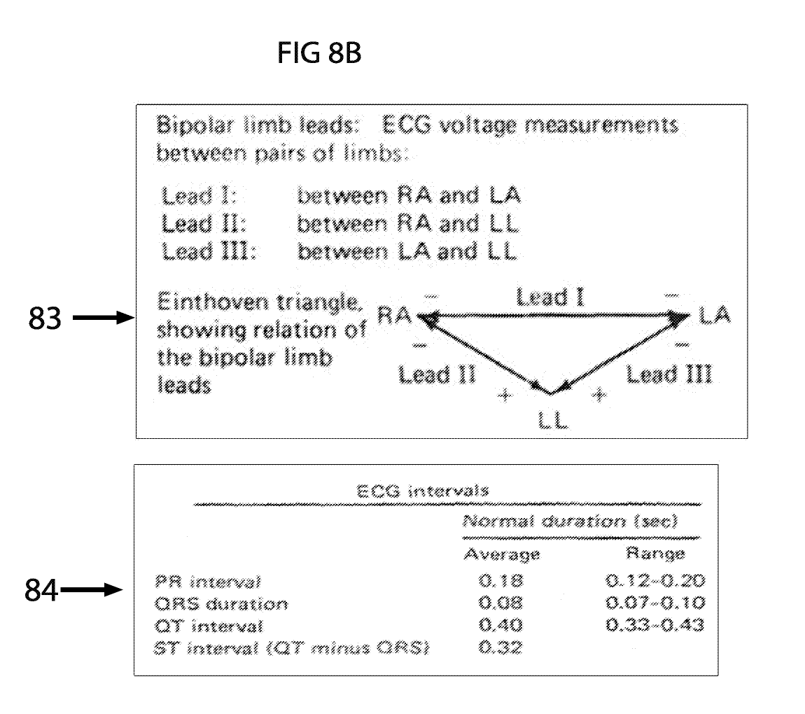

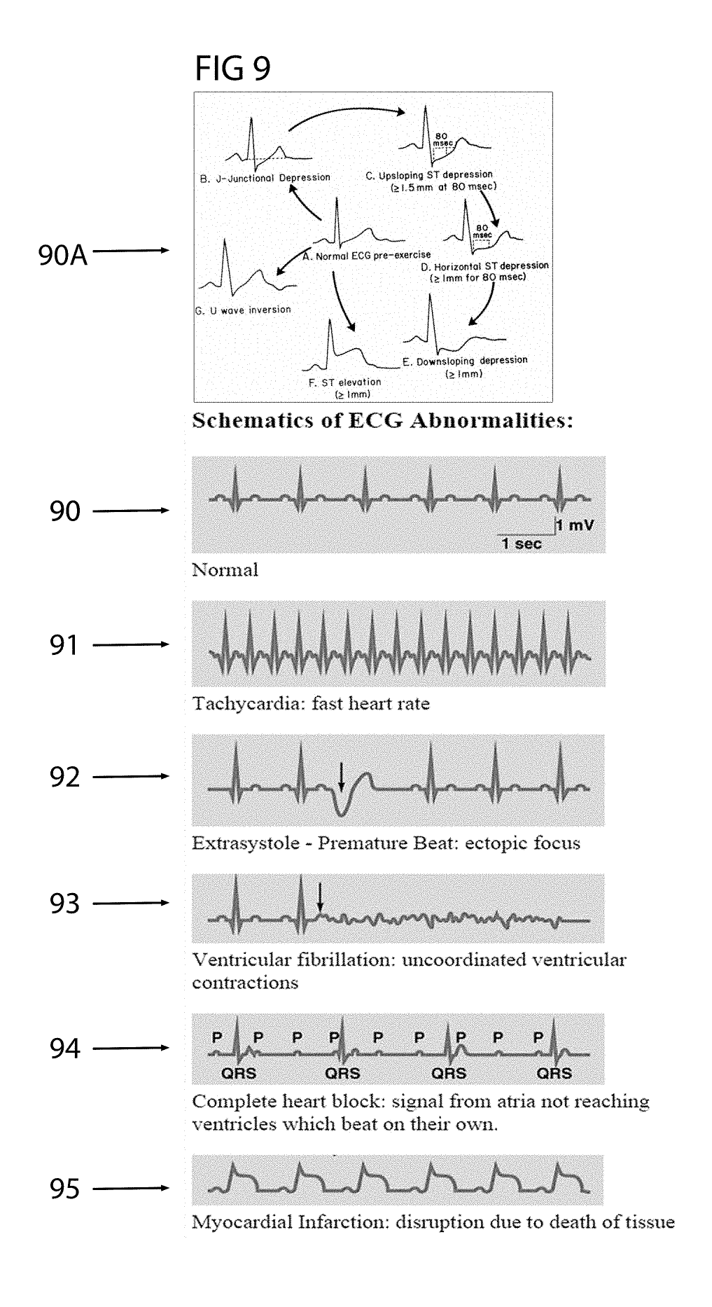

Although the ECG findings of Brugada syndrome were first reported among survivors of cardiac arrest in 1989, it was only in 1992 that the Brugada brothers recognized it as a distinct clinical entity, causing sudden death by causing ventricular fibrillation (FIG. 8 reference number 93)—a lethal arrhythmia—in the heart.

The Artificial Intelligence search, from the ECG streaming sensor (FIG. 1 reference number 15; FIG. 2 reference number 28), for disappearing of the S-T segment. This segment appears form S point and T point. (FIG. 8 reference number 80).

The algorithm of the Artificial Intelligence used to detect the different segment of ECG (FIG. 8 reference number 80) is based on picks detections. It looks for the highest pick one the positive side and negative side. On the positive side this is point R next to it on the right is pick T, next right to T is P. Left to pick R is other P. Left to R there is negative pick Q. And right to R is negative S.

But since in computerize system stream data can arrive from the ECG chip as ADC stream of many samples per second. Only Artificial Intelligence can use old historical streaming as on pick p to determinate backward point as T S R Q P and only then it can find the time gap between the point as segment S-temperature that need to detect Brugada syndrome.

Since this Brugada syndrome can present the disappearing of the S-T segment in rare cases, as one event per few days. Using legacy Holter or legacy while ECG plaster will not detect that. From the simple reason that human doctor can scan and spot signal ECG abnormality is Thousands of ECG samples per day. 86400 samples per day with 60 heart bit per minuets. Only repeated ECG anomaly normally spotted, and at many times is too late.

Only Artificial Intelligence algorithm that is monitored full week can capture the syndrome and prevent the situation become fatal as full ventricular fibrillation FIG. 8 reference number 93 (a lethal arrhythmia) in the heart.

The Artificial Intelligence can also detect on the same 100 msec when ventricular fibrillation happens, and send immediately an alert, with the detected ECG problem. The paramedic can know what medicine to inject patient to recover from Brugada syndrome in ventricular fibrillation state.

FIG. 7 reference number 71—In some cases, the artificial intelligence vital signs wireless plaster can assist paramedics since the Brugada syndrome disease can be detected by observing characteristic patterns on an electrocardiogram, which may be present all the time or might be elicited by the administration of particular drugs (e.g., Class IA (ajmaline) or class 1C (flecainide) antiarrhythmic drugs that block sodium channels and cause appearance of ECG abnormalities) or resurface spontaneously due to as yet un-clarified triggers.

Brugada syndrome has three different ECG patterns.

Type 1 has a coved type ST elevation with at least 2 mm (0.2 mV) J-point elevation a gradually descending ST segment followed by a negative T-wave.

Type 2 has a saddle back pattern with a least 2 mm J-point elevation and at least 1 mm ST elevation with a positive or biphasic T-wave. Type 2 pattern can occasionally be seen in healthy subjects.

Type 3 has either a coved (type 1 like) or a saddle back (type 2 like) pattern with less than 2 mm J-point elevation and less than 1 mm ST elevation. Type 3 pattern is not uncommon in healthy subjects.

FIG. 8 illustrates ECG Electrocardiography.

The ECG records (indirectly) the electrical activity of the heart (FIG. 8 reference number 81). This activity reflects the action of the cardiac muscle as it depolarizes and repolarizes during the cardiac cycle. The ECG represents the temporal and spatial summation of the action potentials of the myocardial fibers typically measured with body-surface electrodes.

ECG's are used to diagnose arrhythmias, abnormal electrolyte (potassium) levels, and conduction abnormalities. They are also used for screening and therapy guidance for heart disease as well as cardiac gathering data for imaging.

In order to get an electrical signal from the body, the plaster has 3 suitable electrodes, amplification and processed. Some cardiac cells generate action potentials (pacemakers). Once generated, and under physiological conditions, the action potential propagates through the cardiac muscle. The temporal and spatial summation of the monophasic action potentials of the myocardial fibers produces an electrical signal known as the ECG

FIG. 8 reference number 80 illustrates Nominal Electrocardiography interval durations

An ECG reflects the sequence of depolarization and repolarization over the contractile chambers of the heart seen using body-surface electrodes. This electrical activity is related to the contraction and relaxation of the heart chambers.

Electrodes measure the voltage between points on the body. A depolarization wavefront (or mean electrical vector) moving toward a positive electrode creates a positive deflection on the ECG in the corresponding lead. A depolarization wavefront moving away from a positive electrode creates a negative deflection in the corresponding lead. A depolarization wavefront moving perpendicular to a positive electrode creates an equi-phasic complex: positive as the depolarization wavefront approaches, negative as it passes.

P wave=atrial depolarization

The PR interval corresponds to the time lag from the onset of atrial depolarization to the onset of ventricular depolarization. This time lag allows atrial systole to occur, filling the ventricles before ventricular systole. Most of the delay occurs in the AV node. A long PR interval corresponds to impaired AV conduction.

QRS complex=ventricular depolarization

The QRS interval represents the time it takes for ventricular depolarization. Normal depolarization requires normal function of the right and left bundle branches. A block in either the right or left bundle branch delays depolarization of the ventricle supplied by the blocked bundle, resulting in a prolonged QRS duration.

T wave=ventricular repolarization

The QT interval represents the time of ventricular depolarization and repolarization. It is useful as a measure of repolarization and is influenced by electrolyte balance, drugs, and ischemia. The QT interval is inversely related to heart rate. A QT interval corrected for heart rate can be calculated.

ECG Acquisition

The plaster electrodes are placed on PCB bottom of the plaster, making it easy to be place to chest.

The electrical signal measured from the surface has amplitude between 10 uV and 5 mV and a bandwidth from 0.05 to 100.00 Hz.

The equipment used to measure and computerized the ECG includes:

Differential amplifier: measures the small voltage difference between two points and tailors the signal into the CPU flash recording and to Artificial Intelligence aggregated buffers.

Isolation: electrically separates the body and the electrical circuits.

filter: removes undesirable signal and improves the signal to noise ratio.

Since the ECG sensor is part of the CPU algorithms, The Artificial Intelligence can reprogram the ECG sensor to optimize samples regarding malefaction and filtering.

FIG. 8 reference number 81 is a Cardiac Vector

The cardiac vector indicates the direction of the depolarization in time. The ECG measured from the pair of the bipolar leads is a time variant, single dimensional projection of the cardiac vector and could be represented using the Einthoven triangle.

FIG. 8—reference numbers 82 and 83 are Einthoven's triangle and the net electrical heart vector.

Einthoven's triangle provides a way to Artificial Intelligence “understand” the amplitude of the ECG waves. One way to verify that ECG data is correct is to plot the cardiac vector into the Einthoven triangle. For this:

1) Construct an equilateral triangle with the base on top. Top will be lead I, right=lead III and left=lead II

2) from the middle point of each side, plot on the corresponding lead (triangle side) a segment of magnitude proportional to the amplitude of the QRS complex.

3) Trace a line perpendicular to the segment at each end of the segment.

The perpendicular lines corresponding to the initial point of the vectors will intersect in the center of the triangle; the perpendiculars traced from the end of the vector should also intersect at one point. The line between the two intersections shows the orientation of the heart. This can explain differences in magnitude between ECG data and the real nominal values.

FIG. 8 reference number 84 is ECG Nominal time Data, this data frame in the base of the Artificial Intelligence to auto recognize the ECG segment

| Wave | Lead I | Lead II | Lead III |
|---|---|---|---|
| P | 0.015 to 0.12 | 0.000 to 0.19 | −0.073 to 0.13 |
| Q | 0.0 to 0.16 | 0.0 to 0.18 | 0.0 to 0.28 |
| R | 0.02 to 1.13 | 0.18 to 1.68 | 0.03 to 1.31 |
| S | 0.0 to 0.36 | 0.0 to 0.49 | 0.0 to 0.55 |
| T | 0.06 to 0.42 | 0.06 to 0.55 | 0.06 to 0.30 |

Nominal range of amplitudes of electrocardiographic waves (mV)

FIG. 8 shows a Schematics of ECG Abnormalities

FIG. 8 reference number 90A—Schematics of ECG Abnormalities, The Artificial Intelligence state machine. Other state machine exists to detect other ECG Abnormalities.

FIG. 8 reference number 91—The Artificial Intelligence detects the time between [R point frequencies and therefore can detect Tachycardia: fast heart rate. In this case the Artificial Intelligence checked the user is in high sport activities and it not, it will send an immediately wireless alert.

FIG. 8 reference number 92—The Artificial Intelligence detects the time between [R point frequencies and therefore can detect missing ECG full segment as present Extrasystole—Premature Beat: ectopic focus

FIG. 8 reference number 93—The Artificial Intelligence detects the time between R point frequencies and therefore can detect missing ECG full segment as present Ventricular fibrillation: uncoordinated ventricular contractions

FIG. 8 reference number 94—The Artificial Intelligence detects full ab—normal ECG activities as in Complete heart block: signal from atria not reaching ventricles which beat on their own.

FIG. 8 reference number 95—The Artificial Intelligence detect full ab—normal ECG activities as in Myocardial Infarction: disruption due to death of tissue

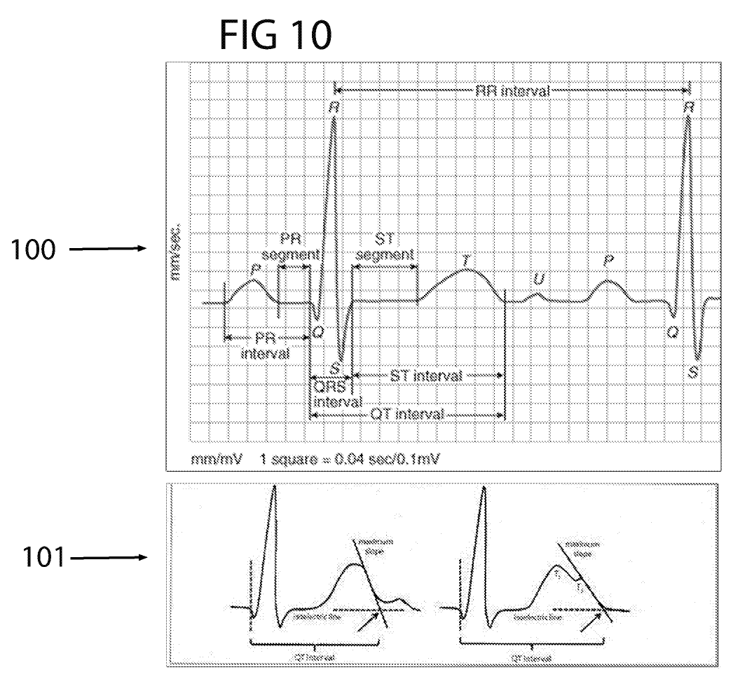

FIG. 10 reference numbers 100 and 101—the Artificial Intelligence algorithms and pattern detection of ECG QT interval for Cardiovascular Disease early detection, Prediction and Prevention. The QT interval FIG. 10 reference number 101 is the time from the start of the Q wave to the end of the T wave. It represents the time taken for ventricular depolarisation and repolarisation.

The Artificial Intelligence uses QT interval as inversely proportional to heart rate:

The QT shortens at faster heart rates

The QT lengthens at slower heart rates

An abnormally prolonged QT is associated with an increased risk of ventricular arrhythmias, especially Torsades de Pointes.

The recently described congenital short QT syndrome has been found to be associated with an increased risk of paroxysmal atrial and ventricular fibrillation and sudden cardiac death.

How the Artificial Intelligence measure QT:

The Artificial Intelligence QT interval measured in from the ECG sensor left and right leads.

Several successive beats is been used be measured, with the maximum interval taken

Large U waves (>1 mm) that are fused to the T wave be included in the measurement.

Smaller U waves and those that are separate from the T wave, the Artificial Intelligence excluded.

The maximum slope intercept method is used to define the end of the T wave FIG. 10 reference number 101

The QT interval is defined from the beginning of the QRS complex to the end of the T wave. The maximum slope intercept method defines the end of the T wave as the intercept between the isoelectric line with the tangent drawn through the maximum down slope of the T wave (left). When notched T waves are present (right), the QT interval is measured from the beginning of the QRS complex extending to the intersection point between the isoelectric line and the tangent drawn from the maximum down slope of the second notch, T2

The Artificial Intelligence corrected QT

The corrected QT interval (QTc) estimates the QT interval at a heart rate of 60 bpm.

This allows comparison of QT values over time at different heart rates and improves Artificial Intelligence detection of patients at increased risk of arrhythmias.

There are multiple formulas used by Artificial Intelligence to estimate QTc (see below).

QTC=QT/√RR Bazett's formula:

QTC=QT/RR⅓ Fredericia's formula:

QTC=QT+0.154(1−RR) Framingham formula:

QTC=QT+1.75(heart rate—60) Hodges formula:

NB. The RR interval is given in seconds (RR interval=60/heart rate).

Normal the Artificial Intelligence uses Bazett's formula due to its simplicity and less CPU power needed. It over-corrects at heart rates >100 bpm and under-corrects at heart rates <60 bpm, but provides an adequate correction for heart rates ranging from 60-100 bpm.

At heart rates outside of the 60-100 bpm range, the Artificial Intelligence uses the Fredericia or Framingham corrections are more accurate and should be used instead.

If an ECG is fortuitously captured while the patient's heart rate is 60 bpm, the absolute QT interval should be used instead!

Normal QTc Values

QTc is prolonged if >440 ms in men or >460 ms in women

QTc>500 is associated with increased risk of torsades de pointes

QTc is abnormally short if <350 ms

A useful rule of thumb is that a normal QT is less than half the preceding RR interval

Causes of a Prolonged QTc (>440 ms)

Hypokalaemia

Hypomagnesaemia

Hypocalcaemia

Hypothermia

Myocardial ischemia

Post-cardiac arrest

Raised intracranial pressure

Congenital long QT syndrome

Hypokalaemia

Hypokalaemia causes apparent QTc prolongation in the limb leads (due to T-U fusion) with prominent U waves in the precordial leads.

Apparent QTc 500 ms—prominent U waves in precordial leads (hypokalaemia (K+ 1.9))

Hypomagnesaemia

QTc 510 ms secondary to hypomagnesaemia

Hypocalcaemia

Hypocalcaemia typically prolongs the ST segment, leaving the T wave unchanged.

QTc 510 ms due to hypocalcaemia

Hypothermia

Severe hypothermia can cause marked QTc prolongation, often in association with bradyarrhythmias (especially slow AF), Osborne waves and shivering artifact.

QTc 620 ms due to severe hypothermia

Myocardial Ischaemia

Myocardial ischemia tends to produce a modest increase in the QTc, in the 450-500 ms range. This may be useful in distinguishing hyperacute MI from benign early repolarization (both may produce similar hyperacute T waves, but BER will usually have a normal QTc).

QTc 495 ms due to hyperacute MI

Raised ICP

A sudden rise in intracranial pressure (e.g. due to subarachnoid haemorrhage) may produce characteristic T wave changes (cerebral T waves′): widespread, deep T wave inversions with a prolonged QTc.

QTc 630 ms with widespread T wave inversion due to subarachnoid haemorrhage

Congenital Long QT Syndrome

There are several congenital disorders of ion channels that produce a long QT syndrome and are associated with increased risk of torsades de pointes and sudden cardiac death.

QTc 550 ms due to congenital long QT syndrome

Causes of a short QTc (<350 ms)

Hypercalcaemia

Congenital short QT syndrome

Digoxin effect

Hypercalcaemia

Hypercalcaemia leads to shortening of the ST segment and may be associated with the appearance of Osborne waves.

Marked shortening of the QTc (260 ms) due to hypercalcaemia

Congenital short QT syndrome

Congenital short QT syndrome (SQTS) is an autosomal-dominant inherited disorder of potassium channels associated with an increased risk of paroxysmal atrial and ventricular fibrillation and sudden cardiac death.

The main ECG changes are very short QTc (<300-350 ms) with tall, peaked T waves.

Very short QTc (280 ms) with tall, peaked T waves due to congenital short QT syndrome

Short QT syndrome may be suggested by the presence of:

Lone atrial fibrillation in young adults

Family member with a short QT interval

Family history of sudden cardiac death

ECG showing QTc<350 ms with tall, peaked T waves

Failure of the QT interval to increase as the heart rate slows

Very short QT (<300 ms) with peaked T waves in two patients with SQTS

Digoxin

Digoxin produces a relative shortening of the QT interval, along with downward sloping ST segment depression in the lateral leads (reverse tick′ appearance), widespread T-wave flattening and inversion, and a multitude of arrhythmias (ventricular ectopy, atrial tachycardia with block, sinus bradycardia, regularized AF, any type of AV block).

Short QT interval due to digoxin (QT 260 ms, QTc 320 ms approx)

QT interval scale

Viskin (2009) proposes the use of a ‘QT interval scale’ to aid diagnosis of patients with short and long QT syndromes (once reversible causes have been excluded):

QT interval scale

Drug-induced QT-Prolongation and Torsades

In the context of acute poisoning with QT-prolonging agents, the risk of TdP is better described by the absolute rather than corrected QT.

More precisely, the risk of TdP is determined by considering both the absolute QT interval and the simultaneous heart rate (i.e. on the same ECG tracing).

These values are then plotted on the QT monogram (below) to determine whether the patient is at risk of TdP.

A QT interval-heart rate pair that plots above the line indicates that the patient is at risk of TdP.

From the monogram, you can see that QTc-prolonging drugs that are associated with a relative tachycardia (e.g. quetiapine) are much less likely to cause TdP than those that are associated with a relative bradycardia (e.g. amisulpride).

There is provided an artificial intelligence vital signs wireless plaster for Cardiovascular Disease early detection, Prediction and Prevention

There may be provided a device for measuring Respiration simultaneously with other sensors.

There may be provided a device for measuring Oxygen in blood monitor simultaneously with other sensors.

There may be provided a device for measuring tremor monitor in movement simultaneously with other sensors.

There may be provided a device for measuring falling monitor simultaneously with other sensors.

There may be provided a device for measuring altitude simultaneously with other sensors.

There may be provided a device for measuring skin temperature simultaneously with other sensors.

There may be provided a device for measuring 3D Accelerometer simultaneously with other sensors.

There may be provided a device that may be configured to detects a gusher hand over above the plaster through clouding simultaneously with other sensors.

There may be provided a device that may be configured to store information in local flash simultaneously with other sensors.

There may be provided a device that may be configured to wireless send to the internet all data and events simultaneously with retrieve data from sensors.

There may be provided a device that may be configured to wireless retrieve from the internet all data, setting and command simultaneously with retrieve data from sensors.

There may be provided a device that may be configured to wireless retrieve new Artificial Intelligence pattern from the internet simultaneously with retrieve data from sensors.

There may be provided a device that may be configured to wireless retrieve new Artificial Intelligence pattern from the internet simultaneously patient's sensor data to test new Artificial Intelligence pattern with real time patient sensors.

There may be provided a device that may be configured to non-wires contact and non-invasive diagnosis and monitoring capabilities of cardiac, pulmonary, and thoracic mechanical functions resulting from normal or induced physiological responses, trauma, disease or response to therapy.

There may be provided a device that may be configured to measuring remotely the physiological parameters of subjects that are fully clothed and that can be either stationary or moving while sitting or standing.

There may be provided a device which can be used as an apnea monitor for patients in hospital or clinic intensive care units, or as a patient monitor in burn or trauma clinics or in nursing homes.

There may be provided a portable device that can be taken into patient areas for the purpose of measuring heart beat and respiration rates.

The additional information may include senses location by proximity to near fix location wireless Bluetooth Smart Wi-Fi bridges, smart phone GPS and send it to internet data base.

The additional information may include patient's hand over the plaster to fire an Event to “call center”, using patient's smart phone or fix location Bluetooth Smart Wi-Fi bridge.

The health monitor may enhance medicine diagnostics.

The health monitor may assist paramedics by observing characteristic patterns on an electrocardiogram.

The health monitor can be update automatized based on reported wireless event or based on time/date.

The health monitor can transmit wirelessly data only on Artificial Intelligence events to extends the battery life immensely and prevents RF radiation of the patient.

The health monitor can be use will patties may be doing heart stress activity to improve the auto learning algorithm.

The wireless transceiver may be use to located patient's in hospital aria.

The health monitor hospital staff or patient's doctor can update setting in the patch regarding margins to an immediate alert using any wireless available channel.

The health monitor may provide a device for non-wires contact and non-invasive diagnosis and monitoring capabilities of cardiac, pulmonary, and thoracic mechanical functions resulting from normal or induced physiological responses, trauma, disease or response to therapy.

The health monitor can receive from a doctor or care giver a command to detect additional physiological and/or additional physiological information such as but not limited to ECG activities, movements, instability, falling, heat, respiration, zone location tracing.

The health monitor may store one or more patterns.

The health monitor may switch between the patterns.

The health monitor may switch between the patterns based on the occurrence of triggers such as but not limited to certain dates and/or times and/or events detected by a currently selected pattern.

The health monitor may receive commands from the person, a doctor or care giver either by a user device, a device accessible to the user or any other device (such as an internet based could computer).

The health monitor may not be capable to generate the patterns—thereby reducing health monitor patch local processing power with outcome reduce the total cost of the health monitor and extend its battery life.

According to an embodiment of the invention the artificial intelligence engine pattern may be also responsive to pattern information. The pattern information may defined pattern values such as physiological information values that may trigger an alert, additional information values that may trigger an event, a combination of multiple values (of physiological information and/or additional information) that may trigger an alert, and the like. Triggering an alert is analogous to an occurrence of an event. For example—a person, a doctor, a medical care giver or any other authorized entity may request to generate an alert when the person is in rest and his heart rate reaches (or exceeds) a certain heart rate value—even if the medical history allows higher heart rate values. If, for example the medical history indicated that the certain heart rate value is too high—then a lower heart rate value (based on the medical history) may or may not be defined as an alert generating event.

Still other objects, features and attendant advantages of the present invention will become apparent to those skilled in the art from a reading of the following detailed description of the preferred embodiment, taken in conjunction with the accompanying drawings.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have,” when used in this specification, specify the presence of stated features, integers, steps, operations, reference number s, and/or components, but do not preclude the presence or addition of one or more other features, integers, steps, operations, reference number s, components, and/or groups thereof.

The corresponding structures, materials, acts, and equivalents of all means plus function reference number s in the claims below are intended to include any structure, or material, for performing the function in combination with other claimed reference number s as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

We claim:

1. A health monitor that comprises:
a patch,
a processor that is attached to the patch,
a wireless transceiver circuit,
one or more physiological sensors for sensing physiological information about a person when the patch is attached to the person, one or more additional sensors for sensing additional environmental information when the patch is attached to the person, and
a memory unit;
wherein the additional information is not physiological information;
wherein the wireless transceiver circuit is configured to wireless transmit a health monitor identifier that identifies the health monitor and to receive, an artificial intelligence engine pattern associated with the person;
wherein the memory unit stores the artificial intelligence engine pattern that (a) is at least partially based on to a medical history of the person; and (b) comprises combinations of values of physiological information and additional environmental information that represent events;
wherein the processor is configured to compare the physiological information and the additional information to the artificial intelligence engine pattern and to trigger alerts upon a detection of an event; and
wherein the wireless transceiver circuit is configured to communicate the alerts.

2. The health monitor according to claim 1 wherein at least a majority of a transmissions of the wireless transceiver circuit are the alerts.

3. The health monitor according to claim 1 wherein the additional information comprises a location of the person.

4. The health monitor according to claim 1 wherein the additional information comprises a detection of an event that includes sensing a gesture of a hand of the person.

5. The health monitor according to claim 4 wherein the gestures triggers a communication with a call center.

6. The health monitor according to claim 1 wherein the wireless transceiver circuit is Bluetooth smart compliant.

7. The health monitor according to claim 1 wherein the wireless transceiver circuit is configured to transmit the physiological information and the additional aggregated stored information.

8. The health monitor according to claim 1 wherein the physiological information is about ECG activities, movements, instability, falling, heat, respiration

9. The health monitor according to claim 1 wherein the wireless transceiver circuit is configured to sense and store in the memory transmissions from wireless identification cards of nurses and doctors.

10. The health monitor according to claim 1 wherein the health monitor is configured to monitor the health monitor battery level and fire alert on low levels.

11. The health monitor according to claim 1 wherein the health monitor is configured to detect patterns of known Cardiovascular Disease

12. The health monitor according to claim 1 wherein the health monitor is configured to detect different person's daily activities and use the right patterns to the activity.

13. The health monitor according to claim 1 wherein the health monitor is configured to monitor the physical information and the additional information while the person performs heart stressing activities and report the physical information and the additional information to an external device

14. The health monitor according to claim 1 wherein the one or more physiological sensor are non-invasive sensors.

15. A method for health monitoring, the method comprises:
transmitting by a wireless transceiver circuit of a health monitor to a communication device a health monitor identifier that identifies the health monitor;
receiving, in response to the health monitor identifier, an artificial intelligence engine pattern associated with a person;
sensing physiological information of a person by one or more physiological sensors while a patch is attached to the person;
sensing by one or more additional sensors additional information about the person while the patch is attached to the person, the additional information is not physiological information;
storing in a memory unit the artificial intelligence engine pattern that (a) is at least partially based on to a medical history of the person and (b) provides alert triggering combinations of values of physiological information and additional information;
comparing, by a processor that is attached to the patch, the physiological information and the additional information to the;
triggering, based on the comparing, alerts; and
communicating the alerts by the wireless transceiver circuit.

16. The method according to claim 15 wherein the additional information comprises environmental information about an environment of the person.

17. A method for health monitoring comprising: calculating, by a computerized system and at least partially based on a medical history of a person, a pattern that (a) is at least partially based on to a medical history of the person and (b) provides alert triggering combinations of values of physiological with additional information; participating in sending the pattern to a health monitor that is associated with the person; receiving from the health monitor, new physiological information of the person and new additional information of the person; updating the pattern to provide an updated pattern; and participating in sending the updated pattern to the health monitor.

18. The method according to claim 17 comprising receiving pattern information that defines pattern values and updating the pattern according to pattern values.

19. The method according to claim 18 wherein the pattern information defines values of at least one of physiological information and additional information that triggers an alert.

20. The method according to claim 18 wherein the pattern information overrides the medical history.
